SEQUENCE LISTING (2 sequences)

SEQ ID NO: 1 - protein, 214 aa, Artificial Sequence
  phage display generated antibody
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 224 aa, Artificial Sequence
  phage display generated antibody
EVQLVESGGGLVQPGGSLRLSCAASGYVFTDYGMNWVRGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

(19) United States
(12) Patent Application Publication
(10) Pub. No.: US 2006/0171940 A1
(43) Pub. Date: Aug. 3, 2006

(54) Antibody disulfide isomers, use thereof, and methods of analyzing same

(75) Inventors: Ned M Mozier, St. Charles, MO (US); Robert L Dufield, Lake Saint Louis, MO (US); Jianming Mo, Chesterfield, MO (US); Gary S Bild, Chesterfield, MO (US)

Correspondence Address: COZEN O'CONNOR, P.C., 1900 MARKET STREET, PHILADELPHIA, PA, 19103-3508, US

(73) Assignee: Celltech R&D Limited, Slough, Berkshire (GB)

(21) Appl. No.: 10/508,711

(22) PCT Filed: Mar. 20, 2003
(86) PCT No.: PCT/US03/08608

§ 371 Date: Jul. 25, 2005

Related U.S. Application Data
(60) Provisional application No. 60/366,350, filed on Mar. 20, 2002.

(51) Int. Cl. A61K 39/395; C07K 16/24

(52) U.S. Cl. 424/133.1; 530/388.23; 424/145.1

(57) ABSTRACT

The present invention relates to disulfide isomers of an antibody having specificity for antigenic determinants of human tumor necrosis factor alpha (TNFα). The present invention also relates to compositions comprising the isomers and therapeutic uses of the antibody. The present invention also relates to analytical methods of detecting the TNFα antibody disulfide isomers and oxidated methionyl forms of the TNFα antibodies.

The present application claims priority under Title 35, United States Code, §119 to U.S. Provisional application Ser. No. 60/366,350, filed Mar. 20, 2002.

### FIELD OF THE INVETION

The present invention relates to recombinant protein disulfide isomers and analytical methods of detecting the disulfide isomers and oxidated methionyl forms of the antibodies. More specifically, it relates to disulfide isomers of an antibody having specificity for antigenic determinants of human tumour necrosis factor alpha (TNFα). The present invention also relates to analytical methods of detecting the TNFα antibody disulfide isomers and oxidated methionyl forms of the TNFα antibodies. The present invention also relates to compositions comprising the isomers and therapeutic uses of the antibody.

### BACKGROND OF THE INVENTION

In an antibody molecule, there are two heavy chains and two light chains. Each heavy chain and each light chain has at its N-terminal end a variable domain. Each variable domain is composed of four framework regions (FRs) alternating with three complementarily determining regions (CDRs). The residues in the variable domains are conventionally numbered according to a system devised by Kabat et al. This system is set forth in Kabat et al, 1987, in Sequences of Proteins of Immunological Interest, US Department of Health and Human Services, NIH, USA (hereafter “Kabat et al. (supra)”). This numbering system is used in the present specification except where otherwise indicated.

The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or CDR, of the basic variable domain structure. The correct Kabat numbering of residues may be determined for a given antibody by alignment of residues of homology in the sequence of the antibody with a “standard” Kabat numbered sequence.

The CDRs of the heavy chain variable domain are located at residues 31-335 (CDRHI), residues 50-65 (CDRH2) and residues 95-102 (CDRH3) according to the Kabat numbering.

The CDRs of the light chain variable domain are located at residues 24-34 (CDRL1), residues 50-56 (CDRL2) and residues 89-97 (CDRL3) according to the Kabat numbering.

Construction of CDR-grafted antibodies is described in European Patent Application EP-A-0239400, which discloses a process in which the CDRs of a mouse monoclonal antibody are grafted onto the framework regions of the variable domains of a human immunoglobulin by site directed mutagenesis using long oligonucleotides. The CDRs determine the antigen binding specificity of antibodies and are relatively short peptide sequences carried on the framework regions of the variable domains.

The earliest work on humanising monoclonal antibodies by CDR-grafting was carried out on monoclonal antibodies recognising synthetic antigens, such as NP.

However, examples in which a mouse monoclonal antibody recognising lysozyme and a rat monoclonal antibody recognising an antigen on human T-cells were humanised by CDR-grafting have been described by Verhoeyen et al. (Science, 239, 1534-1536, 1988) and Riechmann et al. (Nature, 332, 323-324, 1988), respectively.

Riechmann et al., found that the transfer of the CDRs alone (as defined by Kabat (Kabat et al. (supra) and Wu et al., J. Exp. Med., 132, 211-250, 1970)) was not sufficient to provide satisfactory antigen binding activity in the CDR-grafted product. It was found that a number of framework residues have to be altered so that they correspond to those of the donor framework region. Proposed criteria for selecting which framework residues need to be altered are described in International Patent Application WO 90/07861.

A number of reviews discussing CDR-grafted antibodies have been published, including Vaughan et al. (Nature Biotechnology, 16, 535-539, 1998).

TNFα is a pro-inflammatory cytokine that is released by and interacts with cells of the immune system. Thus, TNFα is released by macrophages that have been activated by lipopolysaccharides (LPS) of gram negative bacteria As such, TNFα appears to be an endogenous mediator of central importance involved in the development and pathogenesis of endotoxic shock associated with bacterial sepsis. TNFα has also been shown to be up-regulated in a number of human diseases, including chronic diseases such as rheumatoid arthritis, Crohn's disease, ulcerative colitis, and multiple sclerosis. Mice transgenic for human TNFα produce high levels of TNFα constitutively and develop a spontaneous, destructive polyarthritis resembling rheumatoid arthritis (Kaffer et al., EMBO J., 10, 4025 4031, 1991). TNFα is therefore referred to as a pro-inflammatory cytokine.

Monoclonal antibodies against TNFα have been described in the prior art. Meager et al., (Hybridoma, 6, 305-311, 1987) describe murine monoclonal antibodies against recombinant TNFα. Fendly et al., (Hybridoma, 6 359-370, 1987) describe the use of murine monoclonal antibodies against recombinant TNFα in defining neutralising epitopes on TNF. Shimamoto et al., (Immunology Letters, 17, 311-318, 1988) describe the use of murine monoclonal antibodies against TNF7 and their use in preventing endotoxic shock in mice. Furthermore, in International Patent Application WO 92/11383, recombinant antibodies, including CDR-grafted antibodies, specific for TNFα are disclosed. Rankin et al., (British J. Rheumatology, 34, 334-342, 1995) describe the use of such CDR-grafted antibodies in the treatment of rheumatoid arthritis. U.S. Pat. No. 5,919,452 discloses anti-TNF chimeric antibodies and their use in treating pathologies associated with the presence of 5 TNF.

Antibodies to TNFα have been proposed for the prophylaxis and treatment of endotoxic shock (Beutler et al., Science, 234, 470-474, 1985). Bodmer et al., (Critical Care Medicine, 21, S441-S446, 1993) and Wherry et al., (Critical Care Medicine, 21, S436-S440, 1993) discuss the therapeutic potential of anti-TNFα antibodies in the treatment of septic shock. The use of anti-TNFα antibodies in the treatment of septic shock is also discussed by Kirschenbaum et al., (Critical Care Medicine, 26, 1625-1626, 1998). Collagen-induced arthritis can be treated effectively using an anti-TNFα monoclonal antibody (Williams et al. (PNAS-USA, 89, 9784-9788, 1992)).

Increased levels of TNFα are found in both the synovial fluid and peripheral blood of patients suffering from rheumatoid arthritis. When TNFα blocking agents are administered to patients suffering from rheumatoid arthritis, they reduce inflammation, improve symptoms, and retard joint damage (McKown et al. (Arthritis Rheum., 42, 12041208, 1999).

The use of anti-TNFα antibodies in the treatment of rheumatoid arthritis and 20 Crohn's disease is discussed in Feldman et al., (Transplantation Proceedings, 30, 41264127, 1998), Adorini et al., (Trends in Immunology Today, 18, 209-211, 1997) and in Feldman et al., (Advances in Immunology, 64, 283-350, 1997). The antibodies to TNFα used in such treatments are generally chimeric antibodies, such as those described in U.S. Pat. No. 5,919,452.

Two TNFα blocking products are currently licensed for the treatment of rheumatoid arthritis. The first, called etanercept, is marketed by Immunex Corporation as Enbrel™. It is a recombinant fusion protein comprising two p75 soluble TNF-receptor domains linked to the Fc portion of a human immunoglobulin. The second, called infliximab, is marketed by Centocor Corporation as Remicade™. It is a chimeric antibody having murine anti-TNFα variable domains and human IgG I constant domains.

The prior art recombinant anti-TNFα antibody molecules generally have a reduced affinity for TNFα compared to the antibodies from which the variable regions or CDRs are derived, generally have to be produced in mammalian cells and are expensive to manufacture. Prior art anti-TNFα, antibodies are described in Stephens et al., (Immunology, 85, 668-674, 1995), GB-A-2 246 570 and GB-A-2 297 145.

WO 01/94585 describes antibody molecules having high affinity for TNFα and low immunogenicity in humans, which can be used repeatedly and produced easily and efficiently, to treat chronic inflammatory diseases.

A method for proteolysis in mixed organic-aqueous solvent systems has been described (Russell, WK et al., Anal. Chem. 73:2682-2685, 2001). However, proteolysis of antibody fragments such as Fabs presents unique problems. The difference between the method of the present invention and the method of Russell et al. is the order of addition of the solvents. In the present method, Fab was dissolved in acetonitrile directly and then digestion buffer and enzyme were added. The method of Russell et al. would mix organic solvent and buffer first, then protein and enzyme were added into the solution. However, the method of Russell et al. would not digest Fab efficiently due the compact folded Fab structure.

### BREIF DESCRIPTION OF THE DRAWINGS

FIG. 1 shows the amino acid sequence of the light and heavy chains of CDP870, the intramolecular disulfide pairing, the predominant intermolecular disulfide pairing between the light and heavy chains and the cysteine for PEG conjugation.

FIG. 2 shows the designed disulfide pair Lys-C fragment of CDP870 and the isomer disulfide pair Lys-C fragment of CDP870, where the critical residues (light chain cys-214, heavy chain cys-221 and heavy chain cys-227 are highlighted. The arrows indicate the cleavage pattern of Lys-C.

FIG. 3 shows the methionine containing tryptic fragments of CDP870 in green.

FIG. 4 shows the methionine Oxidation Tryptic Map for CDP870. Tryptic fragments where the methionyl residues are oxidized are described as O1-O5. For example, tryptic fragment M1, when oxidized, is described as O1, M2 as O2 and so on.

FIG. 5 shows the disulfide isomer Lys-C Map for CDP870. The evidence for more abundance 1260.5 amu fragment relative to the 1411.6 amu fragment is apparent.

### SUMMARY OF THE INVENTION

This invention comprises recombinant protein disulfide isomers, preferably, disulfide isomers of an antibody having specificity for antigenic determinants of human tumour necrosis factor alpha (TNFα). The present invention also comprises a method of detecting the antibody disulfide isomers comprising the steps of pre-treating the antibody with an organic solvent, digesting the antibody with a protease, and resolving the fragments. The present invention also relates to analytical methods of detecting the disulfide isomers and oxidated methionyl forms of the TNFα antibodies. The present invention also relates to compositions comprising the isomers and therapeutic uses of the antibody.

### DETAILED DESCRIPTION OF THE INVENTION

In a first aspect, the present invention provides recombinant protein disulfide isomers, having an alternate orientation for disulfide bonds. An “antibody disulfide isomer” is defined as an antibody having disulfide pairing between a cysteine on the light chain and a cysteine on the heavy chain other than the predominant disulfide pairing. Preferably, the disulfide isomer is of an antibody having specificity for antigenic determinants of human tumor necrosis factor alpha (TNFα). Preferably, the TNFα antibody is a TNFα a antibody disclosed in WO 01/04585. Preferably, the antibody is CDP870.

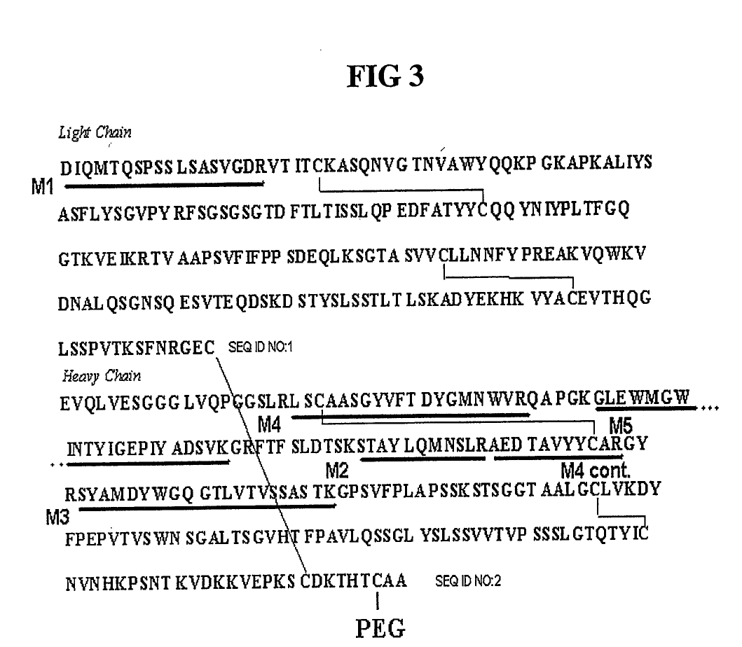

WO 01/04585 provides an antibody molecule having specificity for TNFα, comprising a heavy chain wherein the variable domain comprises a CDR (as defined by Kabat et al., (supra)) having the sequence given as H1 in FIG. 3 of WO 01/04585 (SEQ ID NO: 1 of WO 01/04585) for CDRH1, as H2′ in FIG. 3 of WO 01/04585 (SEQ ID NO: 2 of WO 01/04585) or as H2 in FIG. 3 of WO 01/04585 (SEQ ID NO: 7 of WO 01/04585) for CDRH2 or as H3 in FIG. 3 of WO 01/04585 (SEQ ID NO: 3 of WO 01/04585) for CDRH3.

The antibody molecule of WO 01/04585 comprises at least one CDR selected from H1, H2′ or H2 and H3 (SEQ ID NO: 1; SEQ ID NO: 2 or SEQ ID NO: 7 and SEQ ID NO: 3 of WO 01/04585) for the heavy chain variable domain. Preferably, the antibody molecule comprises at least two and more preferably all three CDRs in the heavy chain variable domain.

In WO 01/04585, there is provided an antibody molecule having specificity for human TNFα, comprising a light chain wherein the variable domain comprises a CDR (as defined by Kabat et al., (supra)) having the sequence given as L1 in FIG. 3 of WO 01/04585 (SEQ ID NO: 4 of WO 01/04585) for CDRL1, L2 in FIG. 3 of WO 01/04585 (SEQ ID NO: 5 of WO 01/04585) for CDRL2 or L3 in FIG. 3 of WO 01/04585 (SEQ ID NO: 6 of WO 01/04585) for CDRL3.

The antibody molecule of WO 01/04585 comprises at least one CDR selected from L1, L2, and L3 (SEQ ID NO: 4 to SEQ ID NO: 6 of WO 01/04585) for the light chain variable domain. Preferably, the antibody molecule comprises at least two and more-preferably all three CDRs in the light chain variable domain.

The antibody molecules of WO 01/04585 preferably have a complementary light chain or a complementary heavy chain, respectively.

Preferably, the antibody molecule of WO 01/04585 comprises a heavy chain wherein the variable domain comprises a CDR (as defined by Kabat et al., (supra)) having the sequence given as H1 in FIG. 3 of WO 01/04585 (SEQ ID NO: 1 of WO 01/04585) for CDRH1, as H2′ or H2 in FIG. 3 of WO 01/04585 (SEQ ID NO: 2 or SEQ ID NO: 7 of WO 01/04585) for CDRH2 or as H3 in FIG. 3 of WO 01/04585 (SEQ ID NO: 3 of WO 01/04585) for CDRH3 and a light chain wherein the variable domain comprises a CDR (as defined by Kabat et al., (supra)) having the sequence given as L1 in FIG. 3 of WO 01/04585 (SEQ ID NO:4 of WO 01/04585) for CDRL1, as L2 in FIG. 3 of WO 01/04585 (SEQ ID NO:5 of WO 01/04585) for CDRL2 or as L3 in FIG. 3 of WO 01/04585 (SEQ ID NO:6 of WO 01/04585) for CDRL3.

The CDRs given in SEQ IDS NOS: 1 and 3 to 7 of WO 01/04585 and in FIG. 3 of WO 01/04585 are derived from a mouse monoclonal antibody hTNF40. However, SEQ ID NO: 2 of WO 01/04585 consists of a hybrid CDR. The hybrid CDR comprises part of heavy chain CDR2 from mouse monoclonal antibody hTNF40 (SEQ ID NO: 7 of WO 01/04585) and part of heavy chain CDR2 from a human group 3 germline V region sequence.

The complete sequences of the variable domains of the mouse hTNF40 antibody are shown in FIGS. 6 of WO 01/04585 (light chain) (SEQ ID NO: 99 of WO 01/04585) and FIG. 7 of WO 01/04585 (heavy chain) (SEQ ID NO: 100 of WO 01/04585). This mouse antibody is referred to below as “the donor antibody”.

A first alternatively embodiment of WO 01/04585 is the mouse monoclonal antibody hTNF40 having the light and heavy chain variable domain sequences shown in FIG. 6 of WO 01/04585 (SEQ ID NO: 99 of WO 01/04585) and FIG. 7 of WO 01/04585 (SEQ ID NO: 100 of WO 01/04585), respectively. The light chain constant region of hTNF40 is kappa and the heavy chain constant region is IgG2a.

In a second alternatively embodiment of WO 01/04585, the antibody according to either of the first and second aspects of WO 01/04585 is a chimeric mouse/human antibody molecule, referred to herein as the chimeric hTNF40 antibody molecule. The chimeric antibody molecule comprises the variable domains of the mouse monoclonal antibody hTNF40 (SEQ ID NOS: 99 and 100 of WO 01/04585) and human constant domains. Preferably, the chimeric hTNF40 antibody molecule comprises the human C kappa domain (Hieter et al., Cell, 22 197-207, 1980; Genebank accession number J00241) in the light chain and the human gamma 4 domains (Flanagan et al., Nature, 300, 709-713, 1982) in the heavy chain.

In a third alternatively embodiment of WO 01/04585, the antibody is a CDR-grafted antibody molecule. The term “a CDR-grafted antibody molecule” as used herein refers to an antibody molecule wherein the heavy and/or light chain contains one or more CDRs (including, if desired, a hybrid CDR) from the donor antibody (e.g. a murine monoclonal antibody) grafted into a heavy and/or light chain variable region framework of an acceptor antibody (e.g. a human antibody).

Preferably, such a CDR-grafted antibody has a variable domain comprising human acceptor framework regions as well as one or more of the donor CDRs referred to above.

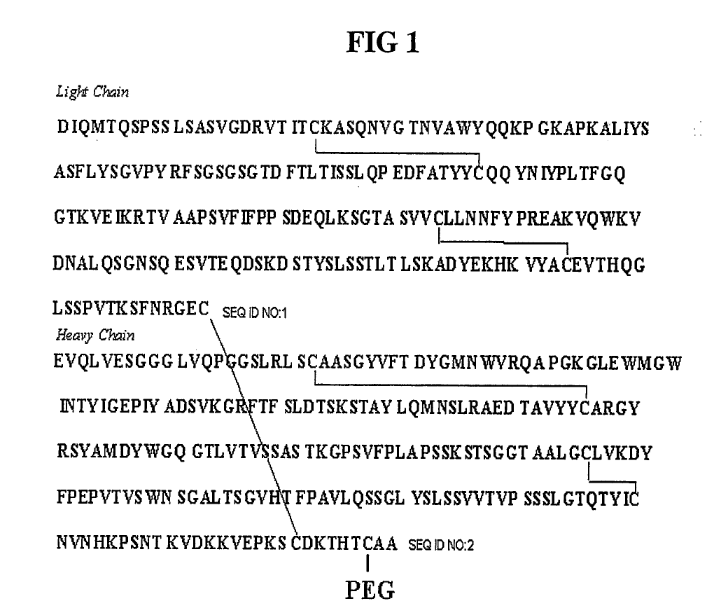

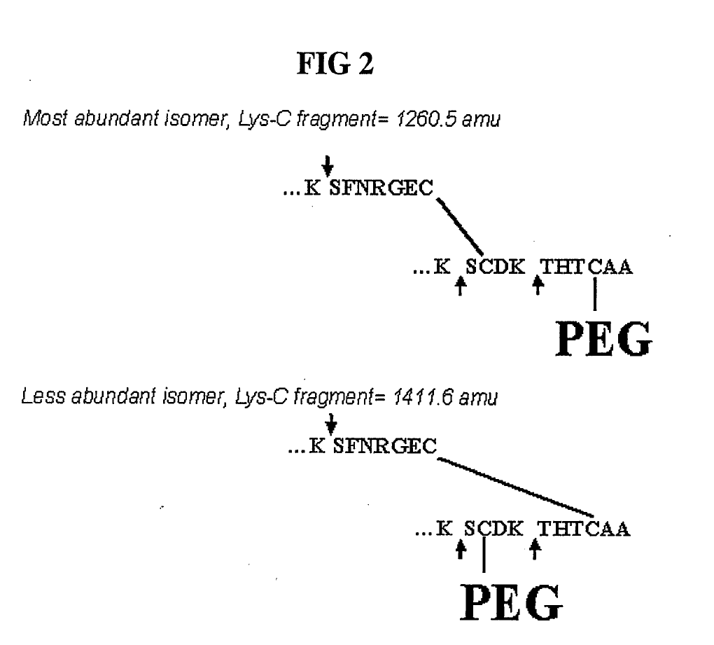

When the CDRs are grafted, any appropriate acceptor variable region framework sequence may be used having regard to the class/type of the donor antibody from which the CDRs are derived, including mouse, primate and human framework regions. Examples of human frameworks of WO 01/04585 are KOL, NEWM, REI, EU, TUR, TEI, LAY, and POM (Kabat et al. (supra)). For example, KOL and NEWM can be used for the heavy chain, REI can be used for the light chain and EU, LAY and POM can be used for both the heavy chain and the light chain. The preferred framework regions for the light chain are the human group 1 framework regions shown in FIG. 1 (SEQ ID NOS: 83, 85, 87 and 89 of WO 01/04585). The preferred framework regions for the heavy chain are the human group 1 and group 3 framework regions shown in FIG. 2 (SEQ ID NOS: 91, 93, 95 and 97 and SEQ ID NOS: 106, 107, 108 and 109 of WO 01/04585), respectively.

In a CDR-grafted antibody of WO 01/04585, it is preferred to use as the acceptor antibody one having chains which are homologous to the chains of the donor antibody. The acceptor heavy and light chains do not necessarily need to be derived from the same antibody and may, if desired, comprise composite chains having framework regions derived from different chains.

Also, in a CDR-grafted antibody of WO 01/04585, the framework regions need not have exactly the same sequence as those of the acceptor antibody. For instance, unusual residues may be changed to more frequently occurring residues for that acceptor chain class or type. Alternatively, selected residues in the acceptor framework regions may be changed so that they correspond to the residue found, at the same position in the donor antibody. Such changes should be kept to the minimum necessary to recover the affinity of the donor antibody. A protocol for selecting residues in the acceptor framework regions which may need to be changed is set forth in WO 91/09967.

Preferably, in a CDR-grafted antibody molecule of the WO 01/04585, if the acceptor heavy chain has human group 1 framework regions (shown in FIG. 2 of WO 01/04585) (SEQ ID NOS: 91, 93, 95 and 97 of WO 01/04585), then the acceptor framework regions of the heavy chain comprise, in addition to one or more donor CDRs, donor residues at positions 28, 69 and 71 (according to Kabat et al. (supra)).

Alternatively, if the acceptor heavy chain has group 1 framework regions, then the acceptor framework regions of the heavy chain comprise, in addition to one or more donor CDRs, donor residues at positions 28, 38, 46, 67, 69, and 71 (according to Kabat et al. (supra).

Preferably, in a CDR-grafted antibody molecule of the WO 01/04585, if the acceptor heavy chain has human group 3 framework regions (shown in FIG. 2 of WO 01/04585) (SEQ ID NOS: 106, 107, 108 and 109 of WO 01/04585), then the acceptor framework regions of the heavy chain comprise, in addition to one or more donor CDRs, donor residues at positions 27, 28, 30, 48, 49, 69,71, 73, 76 and 78 (according to Kabat et al. (supra)).

Preferably, in a CDR-grafted antibody molecule of WO 01/04585 if the acceptor light chain has human group 1 framework regions (shown in FIG. 1 of WO 01/04585) (SEQ ID NOS: 83, 85, 87 and 89 of WO 01/04585) then the acceptor framework regions of the light chain comprise donor residues at positions 46 and 60 (according to Kabat et al. (supra)).

Donor residues are residues from the donor antibody, i.e. the antibody from which the CDRs were originally derived.

The antibody molecule of WO 01/04585 may comprise: a complete antibody molecule, having full length heavy and light chains; a fragment thereof, such as a Fab, modified Fab, Fab′, F(ab′)₂ or Fv fragment; a light chain or heavy chain monomer or dimer; a single chain antibody, e.g. a single chain Fv in which the heavy and light chain variable domains are joined by a peptide linker. Similarly, the heavy and light chain variable regions may be combined with other antibody domains as appropriate.

Preferably the antibody molecule of WO 01/04585 is a Fab fragment. Preferably the Fab fragment has a heavy chain having the sequence given as SEQ ID NO: 111 of WO 01/04585 and a light chain having the sequence given as SEQ ID NO: 113 of WO 01/04585. The amino acid sequences given in SEQ ID NO: 111 and SEQ ID NO: 113 of WO 01/04585 are preferably encoded by the nucleotide sequences given in SEQ ID NO: 110 and SEQ ID NO: 112 of WO 01/04585, respectively.

Alternatively, it is preferred that the antibody molecule of WO 01/04585 is a modified Fab fragment wherein the modification is the addition to the C-terminal end of its heavy chain one or more amino acids to allow the attachment of an effector or reporter molecule. The additional amino acids form a modified hinge region containing one or two cysteine residue to which the effector or reporter molecule may be attached. Such a modified Fab fragment preferably has a heavy chain having the sequence given as SEQ ID NO: 115 of WO 01/04585 and the light chain having the sequence given as SEQ ID NO: 113 of WO 01/04585. The amino acid sequence given in SEQ ID NO: 115 of WO 01/04585 is preferably encoded by the nucleotide sequence given in SEQ ID NO: 114 of WO 01/04585.

A preferred effector group of WO 01/04585 is a polymer molecule, which may be attached to the modified Fab fragment to increase its half-life in vivo.

The polymer molecule may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Particular optional substituents, which may be present on the above-mentioned synthetic polymers, include one or more hydroxy, methyl or methoxy groups. Particular examples of synthetic polymers include optionally substituted straight or branched chain poly(ethyleneglycol), poly(propylene glycol) poly(vinylalcohol) or derivatives thereof, especially optionally substituted poly(ethyleneglycol) such as methoxypoly(ethyleneglycol) or derivatives thereof. Particular naturally occurring polymers include lactose, amylose, dextran, glycogen or derivatives thereof. “Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

The size of the polymer may be varied as desired, but will generally be in an average molecular weight range from 500 Da to 50000 Da, preferably from 5000 to 40000 Da and more preferably from 15000 to 40000 Da. The polymer size may in particular be selected on the basis of the intended use of the product. Thus, for example, where the product is intended to leave the circulation and penetrate tissue, for example for use in the treatment of a tumour, it may be advantageous to use a small molecular weight polymer, for example with a molecular weight of around 5000 Da. For applications where the product remains in the circulation, it may be advantageous to use a higher molecular weight polymer, for example having a molecular weight in the range from 15000 Da to 40000 Da.

Particularly preferred polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

Each polymer molecule attached to the modified antibody fragment may be covalently linked to the sulphur atom of a cysteine residue located in the fragment. The covalent linkage will generally be a disulphide bond or, in particular, a sulphur-carbon bond.

Where desired, the antibody fragment may have one or more effector or reporter molecules attached to it. The effector or reporter molecules may be attached to the antibody fragment through any available amino acid side-chain or terminal amino acid functional group located in the fragment, for example any free amino, imino, hydroxyl or S carboxyl group.

An activated polymer may be used as the starting material in the preparation of polymer-modified antibody fragments as described above. The activated polymer may be any polymer containing a thiol reactive group such as an α-halocarboxylic acid or ester, e.g. iodoacetamide, an imide, e.g. maleimide, a vinyl sulphone, or a disulphide. Such starting materials may be obtained commercially (for example from Shearwater Polymers Inc., Huntsville, Ala., USA) or may be prepared from commercially available starting materials using conventional chemical procedures.

As regards attaching poly(ethyleneglycol) (PEG) moieties, reference is made to “Poly(ethyleneglycol) Chemistry, Biotechnical and Biomedical Applications”, 1992, J. Milton Harris (ed), Plenum Press, New York, “Poly(ethyleneglycol) Chemistry and Biological Applications”, 1997, J. Milton Harris and S. Zalipsky (eds), American Chemical Society, Washington D.C. and “Bioconjugation Protein Coupling Techniques for the Biomedical Sciences”, 1998, M. Aslam and A. Dent, Grove Publishers, New York.

Where it is desired to obtain an antibody fragment linked to an effector or reporter molecule, this may be prepared by standard chemical or recombinant DNA procedures in which the antibody fragment is linked either directly or via a coupling agent to the effector or reporter molecule either before or after reaction with the activated polymer as appropriate. Particular chemical chelating a heavy metal atom, or a toxin, such as ricin, attached to it by a covalent bridging structure. Alternatively, procedures of recombinant DNA technology may be used to produce an antibody molecule in which the Fc fragment (CH2, CH3 and hinge domains), the CH2 and CH3 domains or the CH3 domain of a complete immunoglobulin molecule has (have) been replaced by, or has attached thereto by peptide linkage, a functional non-immunoglobulin protein, such as an enzyme or toxin molecule.

The antibody molecule of WO 01/04585 preferably has a binding affinity of at least 0.85×10⁻¹⁰ M, more preferably at least 0.75×10⁻¹⁰ M and most preferably at least 0.5×10⁻¹⁰ M. (It is worth noting that the preferred humanised antibody molecule of WO 01/04585, as described below, has an affinity of about 0.5×10⁻¹⁰ M, which is better than the affinity of the murine monoclonal antibody from which it is derived. The murine antibody has an affinity of about 0.85×10⁻¹⁰ M.

Preferably, the antibody molecule of WO 01/04585 comprises the light chain variable domain hTNF40-gL1 (SEQ ID NO: 8 of WO 01/04585) and the heavy chain variable domain gh3hTNF40.4 (SEQ ID NO: 11 of WO 01/04585). The sequences of the variable domains of these light and heavy chains are shown in FIGS. 8 and 11 of WO 01/04585, respectively.

WO 01/04585 also relates to variants of the antibody molecule, which have an improved affinity for TNFα. Such variants can be obtained by a number of affinity maturation protocols including mutating the CDRs (Yang et al., J. Mol. Biol., 254, 392403, 1995), chain shuffling (Marks et al., Bio/Technology, 10, 779-783, 1992), use of mutator strains of E. coli (Low et al., J. Mol. Biol., 250, 359-368, 1996), DNA shuffling (Patten et al., Curr. Opin. Biotechnol., 8, 724-733, 1997), phage display (Thompson et al., J. Mol Biol., 256, 77-88, 1996) and sexual PCR (Crameri et al., Nature, 391, 288-291, 1998). Vaughan et al. (supra) discusses these methods of affinity maturation.

WO 01/04585 also provides a DNA sequence encoding the heavy and/or light chain(s) of the antibody molecule.

WO 01/04585 also relates to a cloning or expression vector comprising one or more DNA sequences. Preferably, the cloning or expression procedures include, for example, those described in WO 93/62331, WO 92/22583, WO 90/00195 and WO 89/01476. Alternatively, where the effector or reporter molecule is a protein or polypeptide the linkage may be achieved using recombinant DNA procedures, for example as described in WO 86/01533 and EP-A 392 745.

Preferably, the modified Fab fragment of WO 01/04585 is PEGylated (i.e. has PEG (poly(ethyleneglycol)) covalently attached thereto) according to the method disclosed in EP-A 948 544. Preferably the antibody molecule of WO 01/04585 is a PEGylated modified Fab fragment as shown in FIG. 13 of WO 01/04585. As shown in FIG. 13 of WO 01/04585, the modified Fab fragment has a maleimide group covalently linked to a single thiol group in a modified hinge region. A lysine residue is covalently linked to the maleimide group. To each of the amine groups on the lysine residue is attached a methoxypoly(ethyleneglycol) polymer having a molecular weight of approximately 20,000 Da. The total molecular weight of the entire effector molecule is therefore approximately 40,000 Da These lysine linked PEGs are referred to as “branched PEG” or “U-PEG” as disclosed in U.S. Pat. No. 6,113,906; U.S. Pat. No. 5,919,455; U.S. Pat. No. 5,643,575; and U.S. Pat. No. 5,932,462.

Preferably, in the compound shown in FIG. 13 of WO 01/04585, the heavy chain of the antibody part has the sequence given as SEQ ID NO: 115 of WO 01/04585 and the light chain has the sequence given in SEQ ID NO: 113 of WO 01/04585. This compound is referred to in WO 01/04585 as CDP870.

The constant region domains of the antibody molecule of WO 01/04585, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required, e.g. for simply blocking TNFα activity.

Also, the antibody molecule of WO 01/04585 may have an effector or a reporter molecule attached to it. For instance, it may have a macrocycle, for vector comprises two DNA sequences, encoding the light chain and the heavy chain of the antibody molecule.

WO 01/04585 also relates to host cell/vector systems used for expression of the DNA sequences encoding the antibody molecule. Bacterial, for example E. coli, and other microbial systems may be used, in part, for expression of antibody fragments such as Fab and F(ab′)₂ fragments, and especially Fv fragments and single chain antibody fragments, for example, single chain Fvs. Eukaryotic, e.g. mammalian, host cell expression systems may be used for production of larger antibody molecules, including complete antibody molecules. Suitable mammalian host cells include CHO, myeloma or hybridoma cells.

WO 01/04585 also provides a process for the production of an antibody molecule comprising culturing a host cell comprising a vector under conditions suitable for leading to expression of protein from DNA encoding the antibody molecule, and isolating the antibody molecule.

Preferably the process for the production of the antibody molecule of WO 01/04585 comprises culturing E. coli comprising an E. coli expression vector comprising the DNA sequence under conditions suitable for leading to expression of protein from the DNA sequence and isolating the antibody molecule. The antibody molecule may be secreted from the cell or targeted to the periplasm by suitable signal sequences. Alternatively, the antibody molecules may accumulate within the cell's cytoplasm. Preferably the antibody molecule is targeted to the periplasm. Depending on the antibody molecule being produced and the process used, it is desirable to allow the antibody molecules to refold and adopt a functional conformation. Procedures for allowing antibody molecules to refold are well known to those skilled in the art.

The antibody molecule may comprise only a heavy or light chain polypeptide, in which case only a heavy chain or light chain polypeptide coding sequence needs to be used to transfect the host cells. For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

A preferred embodiment of the present invention is a disulfide isomer of CDP870 having contains a disulfide linkage between Cys 214 of the light chain and Cys 227 of the heavy chain. The predominant linkage that connects the heavy and light chains of CDP870 occurs between Cys 214 of the light chain and Cys 221 of the heavy chain. An unexpected positional disulfide isomer was identified in the CDP870, which contains a disulfide linkage between Cys 214 of the light chain and Cys 227 of the heavy chain.

Another embodiment of the present invention is a therapeutic or diagnostic composition comprising an antibody and at least one disulfide isomer of the antibody in combination with a pharmaceutically acceptable excipient, diluent or carrier. Preferably, the composition comprises CDP870 and at least one disulfide isomer of CDP870 in combination with a pharmaceutically acceptable excipient, diluent or carrier. In a preferred embodiment the composition comprises a CDP870 disulfide isomer, which contains a disulfide linkage between Cys 214 of the light chain and Cys 227 of the heavy chain. In a preferred embodiment of the composition the disulfide isomer comprises between 1% and 60% of the total antibody concentration. In a more preferred embodiment the disulfide isomer comprises between about 10% and 20% of the total antibody concentration. In a more preferred embodiment the disulfide isomer comprises less than about 20% of the total antibody concentration. In a more preferred embodiment the disulfide isomer comprises less than about 15% of the total antibody concentration. In a preferred embodiment the disulfide isomer comprises about 1%, 2%, 3%, 4%, 5%, 6%, 7%, 8%, 9%, 10%, 11%, 12%, 13%, 14%, 15%, 16%, 17%, 18% 19%, or 20% of the total antibody concentration. In a more preferred embodiment the disulfide isomer comprises about 13% of the total antibody concentration. The therapeutic or diagnostic composition or may be accompanied by other active ingredients including other antibody ingredients, for example anti-T cell, anti-IFNY or anti-LPS antibodies, or non-antibody ingredients such as xanthines.

The pharmaceutical compositions should preferably comprise a therapeutically effective amount of the antibody of the invention. The term “therapeutically effective amount” as used herein refers to an amount of a therapeutic agent needed to treat, ameliorate or prevent a targeted disease or condition, or to exhibit a detectable therapeutic or preventative effect. For any antibody, the therapeutically effective dose can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The precise effective amount for a human subject will depend upon the severity of the disease state, the general health of the subject, the age, weight and gender of the subject, diet, time and frequency of administration, drug combination(s), reaction sensitivities and tolerance/response to therapy. This amount can be determined by routine experimentation and is within the judgement of the clinician. Generally, an effective dose will be from 0.01 mg/kg to 50 mg/kg, preferably 0.1 mg/kg to 20 mg/kg, more preferably about 15 mg/kg. As shown in the Examples below, doses of 1, 5 and 20 mg/kg have been used to treat patients suffering from rheumatoid arthritis.

Compositions may be administered individually to a patient or may be administered in combination with other agents, drugs or hormones.

The dose at which the antibody molecule of the present invention is administered depends on the nature of the condition to be treated, the degree to which the level of TNFα to be neutralised is, or is expected to be, raised above a desirable level, and on whether the antibody molecule is being used prophylactically or to treat an existing condition.

Thus, for example, where the product is for treatment or prophylaxis of a chronic inflammatory disease, such as rheumatoid arthritis, suitable doses of the antibody molecule of the present invention lie in the range of between 0.5 and 50 mg/kg, more preferably between 1 and 20 mg/kg and most preferably about 15 mg/kg. The frequency of dose will depend on the half-life of the antibody molecule and the duration of its effect.

If the antibody molecule has a short half-life (e.g. 2 to 10 hours) it may be necessary to give one or more doses per day. Alternatively, if the antibody molecule has a long half life (e.g. 2 to 15 days) it may only be necessary to give a dosage once per day, per week or even once every I or 2 months.

A pharmaceutical composition may also contain a pharmaceutically acceptable carrier for administration of the antibody. The carrier should not itself induce the production of antibodies harmful to the individual receiving the composition and should not be toxic. Suitable carriers may be large, slowly metabolised macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the patient.

Preferred forms for administration include forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilising and/or dispersing agents. Alternatively, the antibody molecule may be in dry form, for reconstitution before use with an appropriate sterile liquid.

Once formulated, the compositions of the invention can be administered directly to the subject. The subjects to be treated can be animals. However, it is preferred that the compositions are adapted for administration to human subjects.

The pharmaceutical compositions of this invention may be administered by any number of routes including, but not limited to, oral, intravenous, intramuscular, intra-arterial, intramedullary, intrathecal, intraventricular, transdermal, transcutaneous (for example, see WO98/20734), subcutaneous, intraperitoneal, intranasal, enteral, topical, sublingual, intravaginal or rectal routes. Hyposprays may also be used to administer the pharmaceutical compositions of the invention. Typically, the therapeutic compositions may be prepared as injectables, either as liquid solutions or suspensions. Solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

It will be appreciated that the active ingredient in the composition will be an antibody molecule. As such, it will be susceptible to degradation in the gastrointestinal tract. Thus, if the composition is to be administered by a route using the gastrointestinal tract, the composition will need to contain agents which protect the antibody from degradation but which release the antibody once it has been absorbed from the gastrointestinal tract.

A thorough discussion of pharmaceutically acceptable carriers is available in Remington's Pharmaceutical Sciences (Mack Publishing Company, N.J. 1991).

The present invention also provides the antibody molecule or compositions of the present invention for use in treating a disease mediated by TNFα.

The present invention further provides the use of the antibody molecule or composition according to the present invention in the manufacture of a medicament for the treatment of a disease mediated by TNFα.

The antibody molecule or composition of the present invention may be utilised in any therapy where it is desired to reduce the level of biologically active TNFα present in the human or animal body. The TNFα may be circulating in the body or present in an undesirably high level localised at a particular site in the body.

For example, elevated levels of TNFα are implicated in acute and chronic immune and immunoregulatory disorders, infections including septic, endotoxic and cardiovascular shock, inflammatory disorders, neurodegenerative diseases, malignant diseases and alcohol induced hepatitis. Details of the numerous disorders associated with elevated levels of TNFα, are set out in U.S. Pat. No. 5,919,452. The antibody molecule or composition of the present invention may be utilised in the therapy of diseases mediated by TNFα. Particularly relevant diseases which may be treated by the antibody molecule of the present invention include sepsis, congestive heart failure, septic or endotoxic shock, cachexia, adult respiratory distress syndrome, AIDS, allergies, psoriasis, TB, inflammatory bone disorders, blood coagulation disorders, bums, rejection episodes following organ or tissue transplant, Crohn's disease and autoimmune diseases, such as thyroiditis and rheumatoid- and osteo-arthritis.

Additionally, the antibody molecule or composition may be used: to reduce side effects associated with TNFα generation during neoplastic therapy; to eliminate or reduce shock-related symptoms associated with the treatment or prevention of graft rejection by use of an anti-lymphocyte antibody; or for treating multi-organ failure.

The antibody molecule or composition of the present invention is preferably used for treatment of rheumatoid- or osteo-arthritis.

The present invention also provides a method of treating human or animal subjects suffering from or at risk of a disorder mediated by TNFα, the method comprising administering to the subject an effective amount of the antibody molecule or composition of the present invention.

The antibody molecule or composition of the present invention may also be used in diagnosis, for example in the in vivo diagnosis and imaging of disease states involving elevated levels of TNFα.

Another embodiment of the present invention is a method of analysis for the characterization and quantitation of disulfide isomers, methionyl oxidations, truncations, deamidation of asparagines, misincorporations, extensions, or other common protein degradations or protein impurities in recombinant proteins. Preferably, the protein is an antibody. More preferably, the antibody has specificity for TNFα. More preferably, the TNFα antibody is CDP870.

The present invention is a novel approach to digesting PEG-antibody fragment for analytical characterization, including but not limited to an Fab, modified Fab, Fab′, F(ab′)₂ or Fv fragment; a light chain or heavy chain monomer or dimer; a single chain antibody, and in this case CDP870. These compounds are very difficult to digest by conventional proteases due to the fact that PEG apparently hinders access of proteolytic enzymes to the protein backbone. This was solved by an assessment of the effect of organic solvents at precise quantities in the reaction mixture. It was discovered that acetonitrile and others were capable of modifying the 3-dimensional structure sufficient to allow complete digestion. Based on this finding, CDP870 could be digested by a number of proteases, including Lys C, and the cleaved protein analyzed by HPLC, mass spectrometry, and other means. This technique was applied to CDP870 and a disulfide isoform was found at approximately 10-20% comprised of material where the C-terminal cysteine residue of light chain was linked to cysteine residue 227 of heavy chain

The method of analysis comprises the steps of pre-treating the recombinant protein with an organic solvent, digesting the pre-treated protein with a protease in the presence of the organic solvent, and a means resolving the protease digest fragments. Preferably, the organic solvent is acetonitrile. Preferably the protease is trypsin for oxidation of methionyl residues and Lys-C for disulfide isomers.

Preferably, the means of resolving is reversed phase HPLC. The method of analysis was used to analyze and quantitate disulfide isomers and methionyl oxidations, but it could also be used for analysis of truncations, deamidation of asparagines, misincorporations, extensions, or other common protein degradations or protein impurities.

The complete content of all publications, patents, and patent applications cited in this disclosure are herein incorporated by reference as if each individual publication, patent, or patent application were specifically and individually indicated to incorporated by reference. Although the foregoing invention has been described in some detail by way of illustration and example for the purposes of clarity of understanding, it will be readily apparent to one skilled in the art in light of the teachings of this invention that changes and modifications can be made without departing from the spirit and scope of the present invention. The following examples are provided for exemplification purposes only and are not intended to limit the scope of the invention, which has been described in broad terms above.

### EXAMPLES

### Example 1

### Method for Detecting Disulfide Isomer

A stock protein solution was prepared by diluting CDP870 to 20 mg/mL or CDP870 Fab′ to 10 mg/mL in 50 mM acetate, 125 mM NaCl, pH 5.5 buffer. Lys-C was purchased from Wako (Osaka, Japan), prepared at 1 mg/mL in 100 mM Tris, pH 8.5.

Pre-Treatment with Acetonitrile

20 μL of diluted sample was mixed with 40 μL acetonitrile, vortexed briefly and let stand for 5 minutes.

Lys-C Digestion

130 μL of 100 mM Tris-HCl, pH 8.5 was added, mixed, then combined with 10 μL Lys-C stock solution. The Lys-C digestion mixture was incubated at 37° C. for about 12-16 hours.

Reversed Phase HPLC

The digest is not quenched and 50 μL was injected directly on the HPLC column (Phenomenex Jupiter 300 angstroms, 5μ, 2.1 mm×250 mm) using a flow rate: 0.5 mL/minute. The Detection Wavelength was 214 nm. The Mobile Phase was A, 0.1% TFA in water and B, 0.1% TFA in acetonitrile. The Gradient (binary system) was as follows:

| Time | Mobile Phase B |
|---|---|
| 0 min. | 0% |
| 3 min | 6% |
| 8 min. | 10% |
| 9 min. | 11% |
| 21 min. | 15% |
| 21.1 min. | 95% |
| 27 min. | 95% |
| 27.1 min. | 0% |

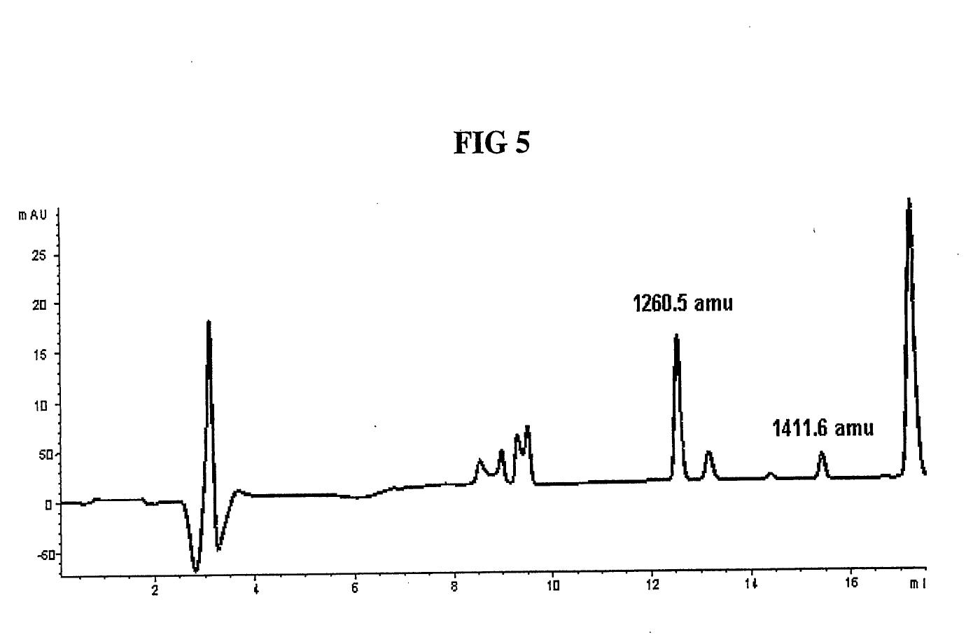

FIG. 5 shows the disulfide isomer Lys-C Map for CDP870

### Example 2

### Method for Detecting Methionine Oxidation

A stock protein solution was prepared by diluting CDP870 to 20 mg/mL or CDP870 Fab′ to 10 mg/mL in 50 mM acetate, 125 mM NaCl, pH 5.5 buffer. Trypsin solution was purchased from Promega (Madison Wis./) with a trypsin concentration of 0.4 mg/mL in 50 mM acetic acid buffer.

Pre-treatment with Acetonitrile

200 μL of diluted sample was mixed with 200 μL acetonitrile, vortexed briefly and let stand for 5 minutes.

Trypsin Digestion

500 μL of digestion buffer (50 mM Tris-HCl, 1 mM CaCl₂, pH 8.5) was added, mixed and then combined with 100 μL trypsin stock solution. The trypsin digestion mixture was incubated at 37° C. for about 12-16 hours.

Reversed Phase HPLC

The digest was quenched with 100 μL 1.0 N HCl and was injected directly on the HPLC column (Zorbax 300SB-C18 (Bodman; Aston, Pa.), 3.5μ, 4.6 mm×15 cm) using a flow rate: 0.5 mL/minute. The Detection Wavelength was 214 nm. The Mobile Phase was A, 0.1% TFA in water and B, 0.1% TFA in acetonitrile. The Gradient (binary system) was as follows:

| Time | Mobile Phase B | |
|---|---|---|
| 0 min. | 16% | |
| 15 min | 25% | Linear |
| 25 min. | 27% | Linear |
| 50 min. | 42% | Linear |
| 52 min. | 90% | Linear |
| 60 min. | 90% | Hold |
| 63 min. | 16% | Linear |
| 78 min. | 16% | Hold |

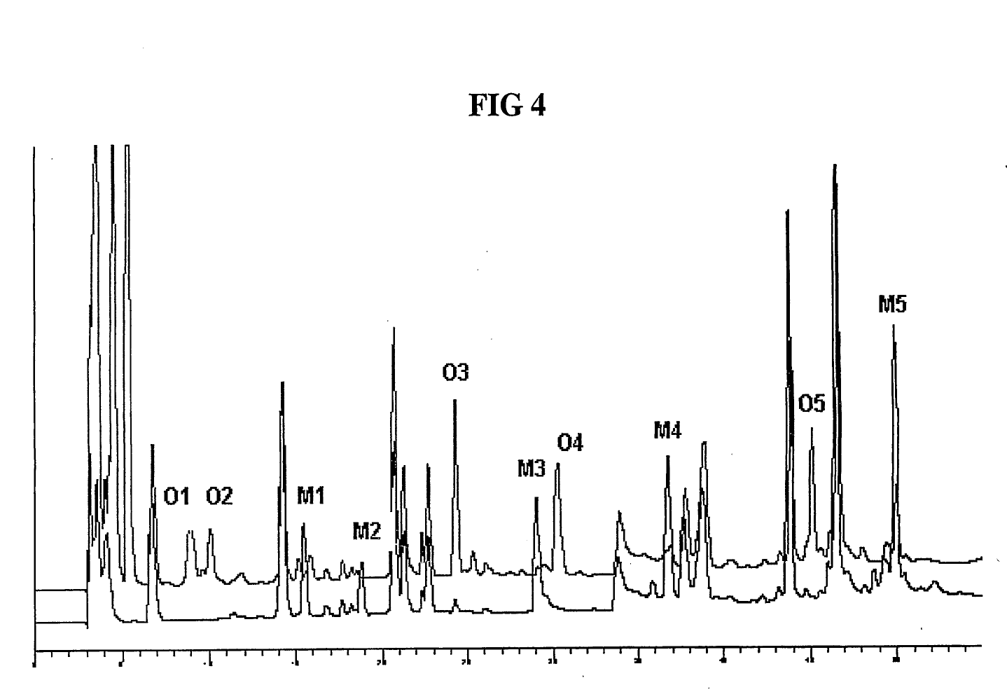

FIG. 4 shows the methionine Oxidation Tryptic Map for CDP870

### Example 3

### TNF Affinity Method

A TNFα affinity column was prepared by coupling human recombinant human TNFα (Biosource International, CA*) to UltraLink™ Biosupport Medium (Pierce, Rockford, Ill.) according to the instructions with the product. TNFα was dissolved in coupling buffer, 0.6 M sodium citrate, 50 mM CHES, pH 9.0. To make a 1 mL column, 0.126 g (dry weight) of UltraLink™ Biosupport was incubated with 750 μL of TNFα in coupling buffer. The reaction was incubated for 72 hours at room temperature with mixing. The beads were centrifuged using an Eppendorf 5415 microfuge at 1000 rpm, 4° C. for 5 minutes. The supernatant was decanted and the resin was resuspended in 10 mL of quench buffer to incubate for 2.5 hours at room temperature in a 15 mL screw top conical with gentle inversion as described previously. Quench buffer was 3 M ethanolamine, pH 9.0. After quenching the conical was centrifuged for 10 minutes (as above), and the supernatant decanted and the resin resuspended in 10 mL of phosphate buffered saline (PBS), pH 7.4 (Invitrogen™ life technologies, Carlsbad, Calif.) for 20 minutes at room temperature with gentle inversion. The washed beads were centrifuged for 10 minutes (as above), the supernatant decanted, and the beads re-suspended in 10 mL of 1 M sodium chloride and incubated for 20 minutes at room temperature with gentle mixing. The beads were centrifuged again, the supernatant decanted, and the beads re-suspended in 10 mL PBS, pH 7.4. This was mixed for 20 minutes, and this step repeated once more. The beads were then re-suspended in 4 mL of PBS, pH 7.4 and poured into a HR 5/5 column. The resulting 1 mL column was equilibrated in 50 column volumes (cv) of buffer A. Buffer A is 10 mM HEPES, 150 mM NaCl pH 7.4.

The column was equilibrated, loaded, washed, eluted, and cleaned at 0.5 ml/min in line on an AKTA Explorer 100 Air with a UV900, pH, temperature, and conductivity meters and the Unicorn 4.0 operating system and analysis software. The column was equilibrated and washed in 10 mM HEPES, 150 mM NaCl, pH 7.4. The column was loaded with CDP870 (36426803, 200 mg/ml stock) that was diluted to 2 mg/mi with Buffer A and injected 0.5 ml/run. The column was eluted isocratically with 100 mM Glycine, pH 3.4. The column was cleaned with 10 mM HEPES, 2 M NaCl, pH 7.4. After loading, the flow-through (unbound) and also the eluted (bound) fractions were collected, and also the eluted fraction (bound). These two fractions and the column feed material were analyzed by LysC digestion followed by mass spectrometry. The peptide map profile showed similar amounts of peptides corresponding to Isomer#1 in all the samples, suggesting that Isomer#1 species were not different than parent in regards to binding TNFα.

We claim:

1. A recombinant protein disulfide isomer.

2. The disulfide isomer of claim 1 wherein said recombinant protein is an antibody.

3. The disulfide isomer of claim 2 wherein said antibody has specificity for TNFα.

4. The disulfide isomer of claim 2 wherein said antibody is a Fab.

5. The disulfide isomer of claim 4 wherein said Fab comprises a modified Fab fragment wherein the modification is the addition to the C-terminal end of its heavy chain one or more amino acids to allow the attachment of an effector or reporter molecule.

6. The disulfide isomer of claim 5 wherein one of said additional amino acid(s) is cysteine.

7. The disulfide isomer of claim 6 wherein said effector is a polymer.

8. The disulfide isomer of claim 7 wherein said polymer is polyethylene glycol.

9. The disulfide isomer of claim 8 wherein said polyethylene glycol is a branched PEG.

10. The disulfide isomer of claim 3 wherein said TNFα antibody is CDP870.

11. The disulfide isomer of claim 10 wherein the disulfide linkage is between Cys 214 of the light chain and Cys 227 of the heavy chain of CDP870.

12. A therapeutic or diagnostic composition comprising a recombinant protein and at least one disulfide isomer of the protein in combination with a pharmaceutically acceptable excipient, diluent, or carrier.

13. The composition of claim 12 wherein said recombinant protein is an antibody.

14. The composition of claim 13 wherein said antibody has specificity for TNFα.

15. The composition of claim 14 wherein said TNFα antibody is CDP870.

16. The composition of claim 15 wherein the disulfide linkage is between Cys 214 of the light chain and Cys 227 of the heavy chain of CDP870.

17. The composition of claim 12, 13, 14, 15, or 16 wherein said disulfide isomer comprises between about 1% and 60% of the total antibody concentration.

18. The composition of claim 17 wherein said disulfide concentration is between about 10% and 20% of the total antibody concentration.

19. The composition of claim 17 wherein said disulfide concentration is less than 20% of the total antibody concentration.

20. The composition of claim 19 wherein said disulfide concentration is less than about 15%.

21. The composition of claim 19 wherein said disulfide concentration is about 13%.

22. A method of analysis for the characterization and quantitation of antibody fragment disulfide isomers comprises the steps of: (a) pre-treating the antibody fragment with an organic solvent, (b) digesting the pre-treated antibody fragment with a proteases in the presence of the organic solvent, and (c) a means resolving the protease digest fragments.

23. The method of claim 22 wherein said organic solvent is acetonitrile.

24. The method of claim 23 wherein said acetonitrile concentration in the pre-treatment step (a) is between about 40% and 80%.

25. The method of claim 24 wherein said acetonitrile concentration in the pre-treatment step (a) is about 67%.

26. The method of claim 23 wherein said acetonitrile concentration in the protease digestion step (b) is between about 20% and 50%.

27. The method of claim 24 wherein said acetonitrile concentration in the protease digestion step (b) is about 20%.

28. The method of claim 22 wherein said protease is Lys-C or trypsin.

29. The method of claim 22 wherein said means resolving the protease digest fragments is reversed phase HPLC.

30. The method of claim 22, 23, 24, 25, 26, 27, 28, or 29 wherein said antibody fragment is selected from the group consisting of: an Fab, modified Fab, Fab′, F(ab′)₂ or Fv fragment; a light chain or heavy chain monomer or dimer; and a single chain antibody.

31. The method of claim 30 wherein said antibody fragment is a Fab.

32. The method of claim 31 wherein said Fab is CD870.

33. A method of analysis for the characterization and quantitation of antibody fragment degradation products and antibody fragment impurities in recombinant proteins selected from the group consisting of methionyl oxidations, truncations, deamidation of asparagines, misincorporations, extensions, or other common comprises the steps of: (a) pre-treating the protein with an organic solvent, (b) digesting the pre-treated protein with a proteases in the presence of the organic solvent, and (c) a means resolving the protease digest fragments.

34. The method of claim 33 wherein said acetonitrile concentration in the pre-treatment step (a) is about 50%.

35. The method of claim 34 wherein said acetonitrile concentration in the protease digestion step (b) is about 20%.
